(19) United States
(12) Patent Application Publication
(10) Pub. No.: US 2009/0270339 A1
(43) Pub. Date: Oct. 29, 2009

(54) Unit Dose Form of Glufosfamide

(76) Inventor: Mike Li, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/303,551

(22) PCT Filed: Jun. 4, 2007
(86) PCT No.: PCT/US07/70351

§ 371 Date: Jun. 24, 2009

Related U.S. Application Data
(60) Provisional application No. 60/811,674, filed on Jun. 6, 2006.

(51) Int. Cl. A61K 31/70; C07H 99/00; C07F 9/547

(52) U.S. Cl. 514/25; 536/17.1; 564/13

(57) ABSTRACT

A lyophilized unit dose form containing about 2 g of glufosfamide is useful in treatment of cancer and other hyper-proliferative diseases.

### CROSS-REFERENCE TO RELATED APPLICATION

This application claims the benefit of U.S. Patent Application No. 60/811,674 filed 6 Jun. 2006, the content of which is incorporated herein by reference.

### FIELD OF THE INVENTION

The present invention provides lyophilized unit dose forms of glufosfamide, an anti-cancer agent, and methods for making them. The invention relates to the fields of chemistry, pharmacology, and medicine.

### BACKGROUND OF THE INVENTION

Glufosfamide, also known as β-D-glucosyl-ifosfamide mustard or glc-IPM, is a prodrug of the alkylator ifosfamide mustard useful in the treatment of cancer (U.S. Pat. No. 5,662,936; PCT App. Pub. No. WO 05/76888; Niculescu-Duvaz, 2002, Curr. Opin. Investig. Drugs, 3:1527-32; Briasoulis et al., 2000, J. Clin. Oncol., 18(20): 3535-44 and 2003, Eur. J. Cancer, 39: 2334-40; Dollner et al., 2004, Anticancer Res., 24(5A):2947-51; and Van der Bent et al., 2003, Ann. Oncol., 14(12):1732-4, each of which is incorporated herein by reference). Glufosfamide is hydrolyzed in vivo to ifosfamide mustard and glucose. In contrast to ifosfamide, glufosfamide metabolism does not produce the neurotoxin acrolein and so promises to have fewer side effects than ifosfamide.

Drugs such as glufosfamide are marketed and made available for administration and sale in a particular form, called the “unit dose form” that contains a specific amount of the drug in a specific formulation. More than one unit dose form can be marketed. The unit dose form of a drug is selected based on, among other factors, the ease of manufacturing the unit dose form, which in turn depends on the cytotoxic and physical characteristics of the drug: the stability of the drug; the therapeutically effective amount of the drug required for a particular type of treatment; and the nature of the drug formulation, e.g., whether the formulation is a powder, a solution, a pill, a tablet, or an emulsion.

Glufosfamide is a cytotoxic solid. Active pharmaceutical ingredient (API) grade glufosfamide obtained as a powder can be placed in a container to yield a solid unit dose form of glufosfamide. However, manufacturing a solid unit dose form of glufosfamide by filling an accurate amount of a powdered form of glufosfamide into a container is difficult as well as hazardous because of the potential for the cytotoxin to become airborne and contaminate the workspace.

The accuracy of filling a particular amount of a solid drug into a container depends on the flow properties of that drug. The flow properties of solid drugs can vary among different batches of the same drug and the amount of a solid drug placed into a given container can therefore vary among different batches. Inaccuracy in the amount of the drug present in unit dose forms of the drug could lead to inaccurate amounts of drug being administered to a patient, leading to ineffective therapy.

Flow problems associated with solid drugs can generally be overcome by dissolving the solid drug in a liquid and dispersing the resulting solution into the unit dose form container, as solutions can generally be dispersed more accurately than solids. If the solid is less stable in solution, or if it is desirable to reduce weight (for lower shipping costs), the liquid can be lyophilized after the solution is dispersed to yield a solid unit dose form. Such a lyophilized form of the API of glufosfamide is known as glufosfamide drug product. Glufosfamide, which has been used in the clinic for several years has been produced in a lyophilized glufosfamide unit dose form containing 1 g of API. Lyophilized glufosfamide is a porous solid and is rehydrated or reconstituted easily into a solution suitable for human administration.

Lyophilization is a process performed in an instrument called a lyophilizer and is used generally for removing water and/or other liquids from aqueous solutions or mixtures at low temperatures of ≦0° C. and involves, among other steps, the step of freezing an aqueous or other solution to a solid form or a frozen mixture; sublimating the ice or other solid under vacuum directly into vapor; and removing the vapor. When lyophilizing an aqueous solution, generally a part of the water is removed by sublimation during a primary drying at a vacuum of V₁ and at temperature of T₁; followed by removal of the residual water by desorption during a secondary drying at a vacuum of V₂ (V₂<V₁) and at temperature of T₂ (T₂>T₁). See for example, Rambhatla et al., 2004, AAPS PharmSciTech., 5 (4): Article 58, incorporated herein by reference. Residual air present during primary drying allows heat transfer via convection and assists in the sublimation. As the lyophilization progresses, a solid product devoid of the liquid accumulates on top of the frozen mixture.

An aqueous solution frozen for lyophilization stays frozen below the eutectic or the glass-transition temperature of the frozen mixture. During lyophilization, if the temperature of the frozen mixture rises above the eutectic temperature or glass transition temperature of the frozen mixture, a melt-back can occur. For a frozen mixture with a low eutectic/glass-transition temperature, a melt-back can also occur during the secondary drying if the primary drying was incomplete. A melt-back during lyophilization of glufosfamide results in inefficient removal of water, a non porous or glassy glufosfamide drug product that cannot be reconstituted as easily as porous glufosfamide, and hydrolytic decomposition of glufosfamide upon storage.

Improper primary drying can occur due to, among other factors, the heterogeneity of ice crystals in the frozen mixture. As currently practiced, the aqueous glufosfamide solution is lyophilized from vials. The sublimation rate of water vapor from ice depends on the size of the ice crystals formed in the vials and the size of pores existing within the frozen mixture. Heterogeneity in ice crystal size in the vials results in heterogeneous rates for freezing-induced sublimation and uneven removal of water among the vials.

Freezing-induced sublimation rate heterogeneity can be reduced by annealing the frozen mixture. Annealing is a process by which a solution or mixture for lyophilization is cooled to a temperature less than or near the eutectic/glass transition temperature of the corresponding frozen mixture for a period of time before the primary drying (Searle et al., 2000, J. Pharm. Sci., 190(7):872-87, incorporated herein by reference).

Lyophilizing glufosfamide solutions can be problematic because the vial size restricts the volume of glufosfamide solution that can be effectively lyophilized from the vial. Currently a 10% (w/v) aqueous solution of glufosfamide is used for lyophilization, requiring about 10 mL of this solution to yield 1 g of lyophilized glufosfamide. Removing more than 10 mL of water by lyophilization from a glufosfamide solution in a vial can lead to impractically long lyophilization cycles and glufosfamide degradation.

As noted above, lyophilized glufosfamide is currently made in unit dose forms of 1 g (a smaller unit dose form of 500 mg has also been made; see, for example, the reference Briasoulis et al., supra). For administration into patients, the 1 g unit dose form of glufosfamide is dissolved in saline, and the resulting solution is administered intravenously to patients. A typical single dose of glufosfamide administered to an adult cancer patient is about 4.5 g/m². Because an adult human has an average surface area of about 1.7 m², the therapeutically effective single dose of glufosfamide administered for treatment of cancer is about 8 g per administration (a single dose of about 8 g is administered no more frequently than once a week). Therefore, the current unit dose form of 1 g is much smaller than the single dose administered to treat cancer, and the administration of glufosfamide to an adult human patient requires reconstitution of about 8 of the currently available unit dose forms.

The administration of glufosfamide would be easier if unit dose forms containing more than 1 g of glufosfamide were available. Thus, there is a need for lyophilized glufosfamide in a unit dose form greater than 1 g, particularly unit dose forms containing 2 g or more of glufosfamide API. The present invention meets this unmet need.

### BRIEF SUMMARY OF THE INVENTION

In one aspect, the present invention provides a lyophilized unit dose form comprising at least about 2 g of glufosfamide API. In one embodiment, the present invention provides a lyophilized unit dose form that contains about 2 g of glufosfamide API. In one embodiment, the lyophilized unit dose form is at least 95% pure. In another embodiment, the lyophilized unit dose form contains no more than 0.5% water.

In another aspect, the present invention provides a container containing about 2 g of lyophilized glufosfamide API. In one embodiment, the container is a glass vial. In one embodiment, the glass vial has a volume of about 50 mL to about 100 mL; an inner diameter of about 3 cm to about 10 cm; and a height of about 10 cm to about 15 cm. In another embodiment, the glass vial has an outer diameter of about 4.3 cm (1.7″) and a height of about 7.6 cm (3.0″). In another embodiment, the glass vial is a molded glass vial. In another embodiment, the glass vial is a tubing glass vial.

In another aspect, the present invention provides a method of producing a lyophilized unit dose form comprising about 2 g of glufosfamide, the method comprising the steps of:

(a) annealing a solution of about 2 g glufosfamide in an aqueous solvent at a temperature of about Tₐ and for a time of about θₐ;

(b) conducting a first primary drying, at a vacuum of about V₁, a temperature of about T₁ₐ, and for a time of about θ_{1a.} of the frozen mixture annealed in step (a);

(c) conducting a second primary drying, at a vacuum of about V₁, a temperature of about T_{1b} wherein T_{1b}>T₁ₐ, and for a time of about θ_{1b.} of the frozen mixture first primary dried in step (b); and

(d) conducting a secondary drying, at a vacuum of about V₂, a temperature of about T₂ wherein T₂>T_{1b}, and for a time of about θ₂, the frozen mixture second primary dried in step (c).

In one embodiment, the annealing temperature, Tₐ, is from about −60° C. to about 5° C. In another embodiment, θₐ is from about 5 h to about 24 h. In another embodiment, V₁ is from about 100 microns to about 200 microns, T₁ₐ is from about −30° C. to about 10° C., θ₁ₐ is from about 60 h to about 80 h, T_{1b} is from about −10° C. to about 5° C., and θ_{1b} is from about 30 h to about 40 h; and V₂ is from about 50 microns to about 90 microns, T₂ is about 25° C., and θ₂ is 20 h to 30 h. In another embodiment, the solution of glufosfamide employed in step (a) is a, (w/v), 5-40%, a 10-20%, or a 10% aqueous solution. In another embodiment, the glufosfamide solution is lyophilized from a container. In another embodiment, the container is a glass vial. In another embodiment, the glufosfamide solution is filled in the vial from about one-third to about one-half of the glass vial height.

In another aspect, the present invention provides a method of treating cancer and other hyperproliferative diseases, said method comprising dissolving a lyophilized unit dose form comprising about 2 g glufosfamide in a pharmaceutically acceptable solvent to yield a pharmaceutically acceptable solution of glufosfamide and administering the pharmaceutically acceptable solution of glufosfamide to a patient in need of such therapy. In one embodiment, the pharmaceutically acceptable solvent is saline.

### DETAILED DESCRIPTION OF THE INVENTION

This detailed description of the different aspects and embodiments of the present invention is organized as follows: Section I provides useful definitions; Section II describes lyophilization of glufosfamide solutions; Section III describes lyophilized unit dose forms containing 2 g glufosfamide: Part A describing compositions, B methods of lyophilization, and C treatment of cancer; and Section IV describes illustrative methods of making a lyophilized unit dose form comprising about 2 g glufosfamide according to the present invention. This detailed description is organized into sections only for the convenience of the reader, and disclosure found in any section is applicable to disclosure elsewhere in the specification.

### I. DEFINITIONS

As used herein, “about” usually means +/−10% of a quantity unless otherwise described in the specification. For example, “about 10 degrees” may mean 10 degrees +/−1 degree (i.e., 9 to 11 degrees), about 2 g may mean from 1.8 to 2.2 g; about 50 mL may mean from 45 to 55 mL, and about 3 cm may mean from 2.7 to 3.3 cm). Alternatively, “about” may be used to refer to a range +/−5% of a quantity. For example, “about 10 degrees” may mean 10 degrees +/−0.5 degrees (i.e., 9.5 to 10.5 degrees), about 2 g may mean from 1.9 to 2.1 g, etc.

“Annealing” refers to a process by which a solution or mixture for lyophilization is cooled to a temperature less than or near the eutectic/glass transition temperature of the corresponding frozen mixture for a period of time before the primary drying (see Searle et al., supra, incorporated herein by reference).

“Administering” or “administration of” a drug to a patient (and grammatical equivalents of this phrase) refers to both to direct administration, including self-administration, and indirect administration, including the act of prescribing a drug. For example, as used herein, a physician who instructs a patient to self-administer a drug and/or provides a patient with a prescription for a drug is administering the drug to the patient.

“Lyophilization” refers to a process used generally for removing water (or other liquids) from frozen aqueous or other liquid containing solutions or mixtures at low temperatures of about ≦0° C. by sublimation and desorption. Lyophilization can be viewed as involving the freezing of the aqueous solution or mixture, thereby converting the water into ice; the sublimating of the ice under vacuum directly into water vapor; and the removing of residual water by desorption. Lyophilization is useful for drying and removing water from substances, and is particularly useful for preparing stable compositions of solids that are susceptible to degradation and/or hydrolysis at about room temperature and higher temperatures.

“Patient” generally refers to a human in need of therapy for the treatment of cancer and other hyperproliferative diseases. “Patient” more broadly refers to any mammal, including non-human primates, suffering from cancer and other hyperproliferative diseases, and non-human mammals used as experimental models of cancer and other hyperproliferative diseases.

A “therapeutically effective amount” of a compound refers to an amount of a compound (drug) that, when administered to a patient with cancer or another hyperproliferative disease, will have the intended therapeutic effect, e.g., alleviation, amelioration, palliation, or elimination of one or more symptoms of cancer or of other hyperproliferative diseases. A “therapeutically effective amount” of a compound may be an amount that, when administered to a patient, delays or slows progression of cancer or other hyperproliferative diseases (compared to expected progression in the absence of treatment). A single therapeutically effective dose of a compound can in some instances be prepared from several unit dose forms of that compound, including a fractional unit dose form. The full therapeutic effect does not necessarily occur by administration of one dose and may occur only after administration of a series of doses. Thus, a therapeutically effective amount can be administered in one or more administrations.

“Treating” a disease, a condition, or a patient refers to taking steps to obtain beneficial or desired results, including clinical results, for the patient. For purposes of this invention, beneficial or desired clinical results include, but are not limited to, prevention, suppression, alleviation, or amelioration of cancer and other hyperproliferative diseases; diminishment of extent of cancer and other hyperproliferative diseases; delay or slowing of progression of cancer and other hyperproliferative diseases; amelioration, palliation, or stabilization of cancer and other hyperproliferative diseases; and other beneficial results.

### II. LYOPHILIZATION

Glufosfamide, an anticancer agent, is lyophilized to produce suitable unit dose forms of glufosfamide drug product. Lyophilization of an aqueous glufosfamide solution depends on a number of factors. Factors affecting lyophilization include, for example, the container or the vial size, the temperatures employed during different stages of the lyophilization, the time period of lyophilization, the concentration of the solution, the rate of cooling, and the like, as described below.

Manufacturing unit dose forms of glufosfamide by lyophilization involves dissolving glufosfamide API in water to yield an aqueous glufosfamide solution. After the aqueous glufosfamide solution is dispersed into vials, the vials are placed on lyophilizer shelves, and the solutions in the vials are frozen to ≦0° C. by shelf-ramp freezing. The process of freezing leads to supercooling. Supercooling, in contrast to a slower gradual cooling of a solution, such as a glufosfamide solution, does not lead to a phase change, such as, for example, freezing of the supercooled solution. A supercooled solution becomes supersaturated, but no solid separates from the liquid phase and no freezing of the solution occurs.

In cases where the supercooling exceeds about 5° C., freezing can take place sequentially by primary nucleation, secondary nucleation encompassing the entire liquid volume, and final solidification. Nucleation temperature heterogeneity during ice formation can result in variation of a morphology-related parameter such as the surface area of the ice crystals, which in turn affects the drying rate and ultimately the lyophilization efficacy.

Supersaturation of glufosfamide solutions during cooling can cause problems in the lyophilization process for other reasons. For example, upon cooling an aqueous glufosfamide solution in vials, water at the bottom of the vial crystallizes to form a frozen mixture that includes ice, and glufosfamide separates out of the solution and forms a layer on top of the frozen mixture. This glufosfamide layer impedes sublimation of water vapor from the underlying ice crystals in the frozen mixture.

To avoid supersaturation of glufosfamide solution upon cooling and separation of solid glufosfamide, a 10% (w/v) glufosfamide solution is employed in the lyophilization. A more concentrated solution increases the possibility of solid glufosfamide separation during shelf-ramp freezing. A more dilute solution can lead to dried product-height related problems, as described below.

To make a lyophilized unit dose form containing about 2 g glufosfamide, about 20 mL of a 10% (w/v) glufosfamide solution is lyophilized, whereas for the 1 g unit dose form, about 10 mL of the same glufosfamide solution is lyophilized. Depending on the diameter of the vials employed in the lyophilization, the glufosfamide solution contained in them will fill the vials to different heights. About 10 and 20 mL of a 10% (w/v) glufosfamide solution in a 4 cm diameter lyophilization vial reach a vial height of about 0.8 cm and about 1.6 cm, respectively. As these solutions undergo primary drying, the height of dried product forming on top of the frozen mixture is taller in the vial containing initially 20 mL of solution than in the one containing 10 mL of solution. During primary drying, the height of dried product forming on top of the frozen mixture affects inversely the rate of sublimation of water vapor from the frozen mixture. See, for example, Rambhatla et al., supra. As a result, the taller the dried product height, the lower the rate of water vapor removal from the underlying frozen mixture, leading to inefficient lyophilization, melt-back, and/or glufosfamide degradation.

If the diameter of the vials containing 20 mL of glufosfamide solution is increased to about 6 cm from about 4 cm, the solution height is reduced to about 0.8 cm. However, only about half the number of 6 cm diameter vials can then be accommodated on the lyophilizer shelves, as compared to 4 cm diameter vials, thereby requiring approximately two lyophilizations to yield the same amount of lyophilized glufosfamide. Increasing the vial diameter thus increases the overall time taken and the cost of lyophilizing the glufosfamide.

The thickness of the glass used in the vials increases as the vial diameter increases. A 6 cm diameter vial is made of glass thicker than that used in a 4 cm diameter vial. The heat transfer rate between the vials and the lyophilization shelf is reduced as the glass in the vial is thickened, for example, as in a molded vial. Slower heat transfer rates can impede the process of freezing and increase lyophilization time and cost.

If removal of water vapor is slowed or prevented during glufosfamide lyophilization, there can be a melt-back during secondary drying. Melt-back results in subsequent formation of non porous or glassy glufosfamide and hydrolytic glufosfamide decomposition.

The present invention provides in part that lyophilization of glufosfamide solution from vials containing about 20 mL of 10% (w/v) glufosfamide (2 g of glufosfamide in each vial) can be performed successfully by annealing the glufosfamide solution at about 0° C. for about 18 h prior to freezing the solution for primary drying. This annealing step allows the ice crystals in the glufosfamide frozen mixture to equilibrate with water. Such equilibration reduces heterogeneity in temperature, ice crystal size, and pore size in the frozen mixture. Thus, this annealing step avoids primary drying rate heterogeneity. By reducing temperature heterogeneity within the frozen mixture, the formation of ice at the vial bottom and subsequent separation of glufosfamide on top of the frozen mixture can be prevented resulting in a process that yields a porous, stable unit dose form containing about 2 g or more of lyophilized glufosfamide.

### III. 2 g LYOPHILIZED UNIT DOSE FORMS OF GLUFOSFAMIDE

### A. Compositions

In one aspect, the present invention provides a lyophilized unit dose form comprising at least about 2 g of glufosfamide API. In one embodiment, the present invention provides a lyophilized unit dose form comprising about 2-4 g of glufosfamide API. In one embodiment, the present invention provides a lyophilized unit dose form comprising about 2.5 g of glufosfamide API. In another embodiment, the present invention provides a lyophilized unit dose form that contains about 2 g of glufosfamide API. In another embodiment, the lyophilized unit dose form contains about 2 g of glufosfamide API and is at least 95% pure. In another embodiment, the lyophilized unit dose form contains no more than 0.5% water. In another embodiment, the lyophilized unit dose form contains no more than 0.5% ifosfamide mustard. In another embodiment, the lyophilized unit dose form contains no more than 0.5% glucose. In one embodiment, the percent of water, ifosfamide mustard, and/or glucose in the unit dose form is determined in comparison with glufosfamide.

In another aspect, the present invention provides a container containing about 2 g of lyophilized glufosfamide API. In one embodiment, the container is a glass vial. In one embodiment, the glass vial has a volume of about 50 mL to about 100 mL; an inner diameter of about 3 cm to about 10 cm; and a height of about 10 cm to about 15 cm. In another embodiment, the glass vial has an outer diameter of about 4.3 cm (1.7″) and a height of about 7.6 cm (3.0″). In another embodiment, the glass vial is a molded glass vial. In another embodiment, the glass vial is a tubing glass vial.

### B. Methods of Lyophilization

In one aspect, the present invention provides a method of producing a lyophilized unit dose form comprising about 2 g of glufosfamide, the method comprising annealing a glufosfamide frozen mixture before the primary drying step. In one embodiment, the primary drying is performed in two stages. In the first stage or the first primary drying, the glufosfamide frozen mixture is subjected to a vacuum of about V₁ and a temp of about T₁ₐ. In a second stage or the second primary drying, the temperature is raised to T_{1b} (T_{1b}>T₁ₐ) while maintaining a vacuum of V₁.

In one embodiment, the present invention provides a method of producing a lyophilized unit dose form comprising about 2 g glufosfamide, the method comprising the steps of:

(a) annealing a solution of about 2 g of glufosfamide in an aqueous solvent at a temperature of about Tₐ and for a time of about θₐ;

(b) conducting a first primary drying, at a vacuum of about V₁, a temperature of about T₁ₐ, and for a time of about θ_{1a,} the frozen mixture annealed in step (a);

(c) conducting a second primary drying, at a vacuum of about V₁, a temperature of about T_{1b} wherein T_{1b}>T₁ₐ, and for a time of about θ_{1b,} the frozen mixture first primary dried in step (b); and

(d) conducting a secondary drying, at a vacuum of about V₂, a temperature of about T₂ wherein T₂>T_{1b}, and for a time of about θ₂, the frozen mixture second primary dried in step (c).

In another embodiment, the annealing temperature, Tₐ, is from about

−60° C. to about 5° C. In another embodiment, θₐ is from about 5 h to about 24 h. In another embodiment, V₁ is from about 100 microns to about 200 microns, T₁ₐ is from about −30° C. to about 10° C., θ₁ₐis from about 60 h to about 80 h, T_{1b} is from about −10° C. to about 5° C., and θ_{1b} is from about 30 h to about 40 h; and V₂ is from about 50 microns to about 90 microns, T₂ is about 25° C., and θ₂ is from about 20 h to about 30 h.

In another embodiment, Tₐ is from about 0° C. and θₐ is about 18 h. In another embodiment, V₁ is from about 120 microns to about 180 microns, T₁ₐ is about −20° C., θ₁ₐ is about 72 h, T_{1b} is about 0° C., θ_{1b} is about 38 h; and V₂ is about 70 microns, T₂ is about 25° C., and θ₂ is about 24 h. In another embodiment, V₁ is about 150 microns.

In another embodiment, the solution of glufosfamide employed in step (a) is a 5-40%, 10-20%, or 10% (w/v) aqueous solution. In another embodiment, the glufosfamide solution is lyophilized from a container. In another embodiment, the container is a glass vial. In another embodiment, the glufosfamide solution is filled in the vial from about one-third to about one-half of the glass vial height.

In another embodiment, T₂ is less than the eutectic temperature of the corresponding glufosfamide water frozen mixture. In one embodiment, T₂ is less than the glass transition temperature of the corresponding glufosfamide water frozen mixture.

### C. Treatment of Cancer and Other Hyperproliferative Diseases

In one aspect, the present invention provides a method of treating cancer and other hyperproliferative diseases, said method comprising dissolving a lyophilized unit dose form comprising about 2 g of glufosfamide in a pharmaceutically acceptable solvent to yield a pharmaceutically acceptable solution of glufosfamide and administering the pharmaceutically acceptable solution of glufosfamide to a patient in need of such therapy. In one embodiment, the pharmaceutically acceptable solvent is saline. In another embodiment, the pharmaceutically acceptable solution of glufosfamide administered to the patient contains about 25 mL to about 1000 mL of saline. Methods for preparing pharmaceutically acceptable solutions are known in the art (see, e.g., Briasoulis et al., Eur. J. Cancer and Briasoulis et al., J. Clin. Oncology, each supra) and can be adapted for use in the present invention by one of skill in the art who has read this disclosure.

In various embodiments, the cancer treated is selected from the group consisting of acute and chronic lymphocytic and granulocytic tumors, adenocarcinoma, adenoma, adrenal cancer, basal cell carcinoma, bone cancer, brain cancer, breast cancer, bronchi cancer, cancer of the larynx, cancer of the colon and/or rectum, cervical dysplasia and in situ carcinoma, epidermoid carcinomas, Ewing's sarcoma, gallbladder, giant cell tumor, hairy-cell tumor, head and neck cancer, hyperplastic corneal nerve tumor, intestinal ganglioneuroma, glioblastoma multiforma, islet cell carcinoma, Kaposi's sarcoma, kidney cancer, liver cancer, leiomyoma, leukemias, lung cancer, lymphomas, malignant carcinoid, malignant hypercalcemia, malignant melanomas, marfanoid habitus tumor, medullary carcinoma, metastatic skin carcinoma, mucosal neuroma, mycosis fungoides, myeloma, neuroblastoma, neural tissue cancer, osteo sarcoma, osteogenic and other sarcoma, ovarian tumor, pancreatic cancer, parathyroid cancer, pheochromocytoma, polycythermia vera, primary brain tumor, prostate cancer, renal cell tumor, retinoblastoma, rhabdomyosarcoma, seminoma, skin cancer, small-cell lung tumor, soft tissue sarcoma, squamous cell carcinoma of both ulcerating and papillary type, stomach cancer, topical skin lesion, thyroid cancer, veticulum cell sarcoma, and Wilm's tumor.

In certain embodiments, the cancer treated is selected from the group consisting of pancreatic cancer, Gemzar™ resistant pancreatic cancer, small cell lung cancer, non Hodgkin's lymphoma, sarcoma, and adriamycin resistant sarcoma. In certain other embodiments, the cancer treated is selected from the group consisting of breast cancer, ovarian cancer, and colorectal cancer. Treatment of various cancers in accordance with the present method can be adapted by one of skill who has read this disclosure from the methods described for example in PCT App. Pub. Nos. WO 05/76888 (supra); WO 06/071955; WO 06/122227; and WO 07/035961; and the reference Niculescu-Duvaz (supra), each of which is incorporated herein by reference.

In another aspect, the present invention provides a method of treating non-cancer hyperproliferative diseases characterized by cellular hyperproliferation (e.g., an abnormally increased rate or amount of cellular proliferation). In certain embodiments, the hyperproliferative disease treated according to the present method is selected from the group consisting of allergic angiitis and granulomatosis (Churg-Strauss disease), asbestosis, asthma, atrophic gastritis, benign prostatic hyperplasia, bulbs pemphigoid, coeliac disease, chronic bronchitis and chronic obstructive airway disease, chronic sinusitis, Crohn's disease, demyelinating neuropathies, dermatomyositis, eczema including atopic dermatitis, eustachean tube diseases, giant cell arteritis, graft rejection, hypersensitivity pneumonitis, hypersensitivity vasculitis (Henoch-Schonlein purpura), irritant dermatitis, inflammatory hemolytic anemia, inflammatory neutropenia, inflammatory bowel disease, Kawasaki's disease, multiple sclerosis, myocarditis, myositis, nasal polyps, nasolacrimal duct diseases, neoplastic vasculitis, pancreatitis, pemphigus vulgaris, primary glomerulonephritis, psoriasis, periodontal disease, polycystic kidney disease, polyarteritis nodosa, polyangitis overlap syndrome, primary sclerosing cholangitis, rheumatoid arthritis, serum sickness, surgical adhesions, stenosis or restenosis, scleritis, scleroderma, strictures of bile ducts, strictures (of duodenum, small bowel, and colon), silicosis and other forms of pneumoconiosis, type I diabetes, ulcerative colitis, ulcerative proctitis, vasculitis associated with connective tissue disorders, vasculitis associated with congenital deficiencies of the complement system, vasculitis of the central nervous system, and Wegener's granulomatosis.

In one embodiment, the hyperpriliferative disease treated is psoriasis, a disease characterized by the cellular hyperproliferation of keratinocytes which builds up on the skin to form elevated, scaly lesions. In another embodiment, the hyperproliferative disease treated is multiple sclerosis, a disease characterized by progressive demyelination in the brain. In another embodiment, the hyperproliferative diseases treated is rheumatoid arthritis, a multisystem chronic, relapsing, inflammatory disease that can lead to destruction and ankyiosis of joints affected. In another embodiment, the hyperproliferative disease treated is benign prostatic hyperplasia, a disease in which prostate epithelial cells grow abnormally and thereby block urine flow.

The invention, having been described in summary and in detail, is illustrated but not limited by the examples below, which demonstrate methods of making lyophilized unit dose forms containing about 2 g of glufosfamide API.

### IV. EXAMPLES

### Example 1

### Lyophilization of Aqueous Glufosfamide Solution

Glufosfamide (630 g) was accurately weighed in a 10-L glass beaker and dissolved in sterile water for injection to yield a clear solution (Formulation A, 6300 mL, 6502 g). The temperature of Formulation A was measured to be 20° C.

Formulation A was filtered through a 0.22-μm filter, and 20.5±0.5 mL of the filtrate added to about one hundred and fifty 50 mL vials using a Flexicon filling machine. The vials were stoppered with lyophilization stoppers and placed in a Hull 24 lyophilizer with a shelf temperature of 0° C. The temperature of the contents of the vials was determined by placing thermocouples into two vials.

The shelf temperature was reduced to −35° C. and maintained at that temperature for 3 hours, following which the contents of the vials were annealed by raising the shelf temperature to 0° C. and maintaining the temperature at 0° C. for 18 hours. The shelf temperature was then lowered to −45° C. and raised to

−20° C. The condenser (<50° C.) was turned on, and the contents of the vials were lyophilized under vacuum (150±30 micron) for 72 hours, following which the shelf temperature was raised to 0° C. and lyophilization continued for 38 hours. The shelf temperature was then raised to 25° C. over a period of 1 hour and the lyophilization continued at 70 microns for 24 hours. The lyophilization was terminated by allowing nitrogen into the chamber and sealing the vials under partial vacuum (9-11 psi) to yield a unit dose form containing about 2 g of glufosfamide. The vials where stored in a refrigerator and later assayed (by HPLC) and determined to contain 99.2% glufosfamide. After 2 weeks at 25° C. and 60% relative humidity, this unit dose form was assayed (by HPLC) to contain 98% glufosfamide. Thus, this example demonstrates making a lyophilized unit dose form containing about 2 g glufosfamide in accordance with the methods of the present invention.

### Example 2

### Lyophilization of Aqueous Glufosfamide Solution with Mannitol

Formulation A (3251 g, supra) was transferred into a 4-L glass beaker followed by the addition of mannitol (31.5 g) to yield Formulation B. Formulation B was lyophilized as described above for Formulation A to yield a lyophilized unit dose form of about 2 g of glufosfamide which was assayed (by HPLC) to contain 97.6% glufosfamide. After 2 weeks at 25° C. and 60% relative humidity, this lyophilized unit dose form was assayed (by HPLC) to contain glufosfamide which is about 94.8% pure.

Although the present invention has been described in detail with reference to specific embodiments, those of skill in the art will recognize that modifications and improvements are within the scope and spirit of the invention, as set forth in the claims that follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of publication of the same. The invention having now been described by way of written description and example, those of skill in the art will recognize that the invention can be practiced in a variety of embodiments and that the foregoing description and examples are for purposes of illustration and not limitation of the following claims.

1. A lyophilized unit dose form comprising at least about 2 g of glufosfamide API.

2. The lyophilized unit dose form of claim 1 that contains about 2 g of glufosfamide API.

3. The lyophilized unit dose form of claim 1 that is at least 95% pure.

4. The lyophilized unit dose form of claim 1 containing no more that 0.5% water.

5. The lyophilized unit dose form of claim 1 containing no more that 0.5% ifosfamide mustard.

6. The lyophilized unit dose form of claim 1 containing no more that 0.5% glucose.

7. The lyophilized unit dose form of claim 1 wherein the unit dose form is contained in a glass vial.

8. The lyophilized unit dose form of claim 7, wherein the container is a glass vial having a volume of about 50 mL to about 100 mL; an inner diameter of about 3 cm to about 10 cm; and a length of about 10 cm to about 15 cm.

9. A method of producing a lyophilized unit dose form comprising about 2 g of glufosfamide, the method comprising the steps of:
(a) annealing a solution of about 2 g glufosfamide and an aqueous solvent at a temperature of about Tₐ and for a time of about θₐ;
(b) conducting a first primary drying, at a vacuum of about V₁, a temperature of about T₁ₐ, and for a time of about θ_{1a.} the frozen mixture annealed in step (a);
(c) conducting a second primary drying, at a vacuum of about V₁, a temperature of about T_{1b} wherein T_{1b}>T₁ₐ, and for a time of about θ_{1b}, the frozen mixture first primary dried in step (b); and
(d) conducting a secondary drying, at a vacuum of about V₂, a temperature of about T₂ wherein T₂>T_{1b}, and for a time of about θ₂, the frozen mixture second primary dried in step (c)
wherein, Tₐ is about 0° C. and θₐ is about 18 h; V₁ is from about 120 microns to about 180 microns, T₁ₐ is about −20° C., θ₁ₐ is about 72 h, T_{1b} is about 0° C., θ_{1b} is about 38 h; and V₂ is about 70 microns, T₂ is about 25° C., and θ₂ is about 24 h.

10. The method of claim 9 for producing a lyophilized unit dose form containing about 2 g of glufosfamide.

11. A method of treating cancer, said method comprising dissolving a lyophilized unit dose form comprising about 2 g of glufosfamide in a pharmaceutically acceptable solvent to yield a pharmaceutically acceptable solution of glufosfamide and administering the pharmaceutically acceptable solution of glufosfamide to a patient in need of such therapy.

12. The method of claim 11 wherein the pharmaceutically acceptable solvent is saline.
